(19) United States
(12) Patent Application Publication
(10) Pub. No.: US 2012/0220969 A1
(43) Pub. Date: Aug. 30, 2012

(54) HEALTH MONITORING APPARATUS AND METHOD

(76) Inventors: Seungjin JANG, Anyang (KR); Youngdon Hwang, Seoul (KR); Jeongmee Koh, Seoul (KR); Younghyeog Jeon, Seoul (KR)

(21) Appl. No.: 13/035,709

(22) Filed: Feb. 25, 2011

(51) Int. Cl. A61F 13/42

(52) U.S. Cl. 604/361

(57) ABSTRACT

A health monitoring apparatus for determining whether or not a diaper wearer has excreted based on spectrum information detected from an acoustic signal generated from the diaper, and monitoring the diaper wearer's physical condition according to the characteristics of the body wastes through color pattern-based image processing, and a health monitoring method using the same are provided. The health monitoring method of a health monitoring apparatus includes: receiving an acoustic signal from a sensing device attached to a diaper; determining whether any body wastes have been discharged based on a spectrum extracted from the acoustic signal; when it is determined that body wastes have been discharged, obtaining an image of the diaper by using a camera; obtaining characteristic information of the body wastes based on a color pattern extracted from the obtained image; and transmitting information regarding the diaper wearer's physical condition to an external terminal based on the characteristic information.

### BACKGROUND OF THE INVENTION

1. Field of the Invention

The present disclosure relates to a health monitoring apparatus and method and, more particularly, to a health monitoring apparatus for monitoring diaper wearers' physical condition and a health monitoring method in the apparatus.

2. Description of the Related Art

Recently, a sensing technique that measures information relevant to a user's physical condition has been advancing, so an apparatus for conveniently monitoring a user's physical condition by using such a sensing technique is requested to be considered.

### SUMMARY OF THE INVENTION

An aspect of the present disclosure provides a health monitoring apparatus for determining whether or not a diaper wearer has excreted based on spectrum information detected from an acoustic signal generated from the diaper, and monitoring the diaper wearer's physical condition according to characteristic information of the body wastes through color pattern-based image processing, and a health monitoring method using the same.

According to an aspect of the present invention, there is provided a health monitoring method of a health monitoring apparatus, including: receiving an acoustic signal from a sensing device attached to a diaper; determining whether any body wastes have been discharged based on a spectrum extracted from the acoustic signal; when it is determined that body wastes have been discharged, obtaining an image of the diaper by using a camera; obtaining characteristic information of the body wastes based on a color pattern extracted from the obtained image; and transmitting information regarding the diaper wearer's physical condition to an external terminal based on the characteristic information.

The spectrum may include a wideband spectrum and/or narrowband spectrum extracted from the acoustic signal.

In determining whether any body wastes have been discharged, whether any body wastes have been discharged may be determined based on a distribution range of a basic frequency on the wideband spectrum.

In determining whether any body wastes have been discharged, whether any body wastes have been discharged may be determined based on a distribution range of a maximum frequency of the narrowband spectrum.

The method may further include: receiving a trigger signal from the sensing device, wherein, in obtaining the image of the diaper, the image of the diaper may be obtained based on the trigger signal.

The method may further include: transmitting information regarding a point in time at which the trigger signal was received to the external terminal.

The trigger signal may be generated when the sensing device is separated from the diaper.

The obtaining of the characteristic information of body wastes may include: detecting an area corresponding to the diaper from the obtained image; and determining whether or not the body wastes are excrements or urine based on a color contrast or turbidity of the detected area.

The obtaining of the image may include: detecting illuminance information of surroundings; and controlling illumination based on the detected illuminance information.

The obtaining of the image may include: when it is determined that body wastes have been discharged, transmitting a message informing that body wastes have been discharged to the external terminal.

According to another aspect of the present invention, there is provided a health monitoring apparatus including: a communication unit configured to receive an acoustic signal from a sensing device attached to a diaper; a camera configured to acquire an image of the diaper; and a controller configured to determine whether or not any body wastes have been discharged based on a spectrum extracted from the acoustic signal, control the camera to acquire the image when it is determined that body wastes have been discharged, obtain characteristic information of the body wastes based on a color pattern extracted from the obtained image, and control the communication unit to transmit obtained information regarding the diaper wearer's physical condition to an external terminal based on the characteristic information.

The spectrum may include a wideband spectrum and/or narrowband spectrum extracted from the acoustic signal.

The controller may determine whether any body wastes have been discharged, based on a distribution range of a basic frequency on the wideband spectrum.

The controller may determine whether any body wastes have been discharged is determined, based on a distribution range of a maximum frequency of the narrowband spectrum.

The communication may receive a trigger signal from the sensing device, and the controller may control the camera to obtain the image based on the trigger signal.

The communication unit may transmit information regarding a point in time at which the trigger signal was received to the external terminal.

The trigger signal may be generated when the sensing device is separated from the diaper.

The controller may detect an area corresponding to the diaper from the obtained image, and determine whether or not the body wastes are excrements or urine based on a color contrast or turbidity of the detected area.

The health monitoring apparatus may further include: an illumination unit providing illumination by using an illumination source; and an illuminance sensing device configured to detect illuminance information of surroundings, and the controller may control illumination provided by the illumination unit based on the detected illuminance information.

When it is determined that body wastes have been discharged, the controller may control the communication unit to transmit a message informing that body wastes have been discharged, to the external terminal.

Further scope of applicability of the present invention will become apparent from the detailed description given hereinafter. However, it should be understood that the detailed description and specific examples, while indicating preferred embodiments of the invention, are given by illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings, which are given by illustration only, and thus are not limitative of the present invention, and wherein:

FIG. 1 is a view illustrating the configuration of a health monitoring system according to an exemplary embodiment of the present invention;

FIG. 2 is a schematic block diagram of a sensing device according to an exemplary embodiment of the present invention;

FIG. 3 is a schematic block diagram of a health monitoring apparatus according to an exemplary embodiment of the present invention;

FIG. 4 is a flow chart illustrating a health monitoring process of the health monitoring apparatus according to an exemplary embodiment of the present invention;

FIG. 5 is a view for explaining spectrum information extracted from an acoustic signal received by the health monitoring apparatus according to an exemplary embodiment of the present invention from the sensing device;

FIGS. 6A to 6D are views for explaining color patterns extracted from images obtained by the health monitoring apparatus according to an exemplary embodiment of the present invention;

FIG. 7 is a view for explaining a color pattern extracted from an image obtained by the health monitoring apparatus according to an exemplary embodiment of the present invention;

FIG. 8A is a view for explaining a process of receiving an acoustic signal or a trigger signal from the sensing device by the health monitoring apparatus according to an exemplary embodiment of the present invention;

FIG. 8B is a view for explaining a process of analyzing a color pattern from an image of a diaper obtained by the health monitoring apparatus according to an exemplary embodiment of the present invention;

FIG. 9 is an overview of a display screen illustrating a transmission of a message indicating that body wastes have been discharged to an external terminal by the health monitoring apparatus according to an exemplary embodiment of the present invention; and

FIGS. 10A and 10B are overviews of display screens illustrating the process of transmitting health information to the external terminal by the health monitoring apparatus according to an exemplary embodiment of the present invention.

### DETAILED DESCRIPTION OF THE INVENTION

A health monitoring apparatus and a health monitoring method thereof according to exemplary embodiments of the present invention will now be described with reference to the accompanying drawings. In the following description, usage of suffixes such as ‘module’, ‘part’ or ‘unit’ used for referring to elements is given merely to facilitate explanation of the present invention, without having any significant meaning by itself. The terms used in the present disclosure are used for facilitate explanation of the exemplary embodiments of the present invention. Thus, the exemplary embodiments of the present invention are not limited to the terms used in the present disclosure.

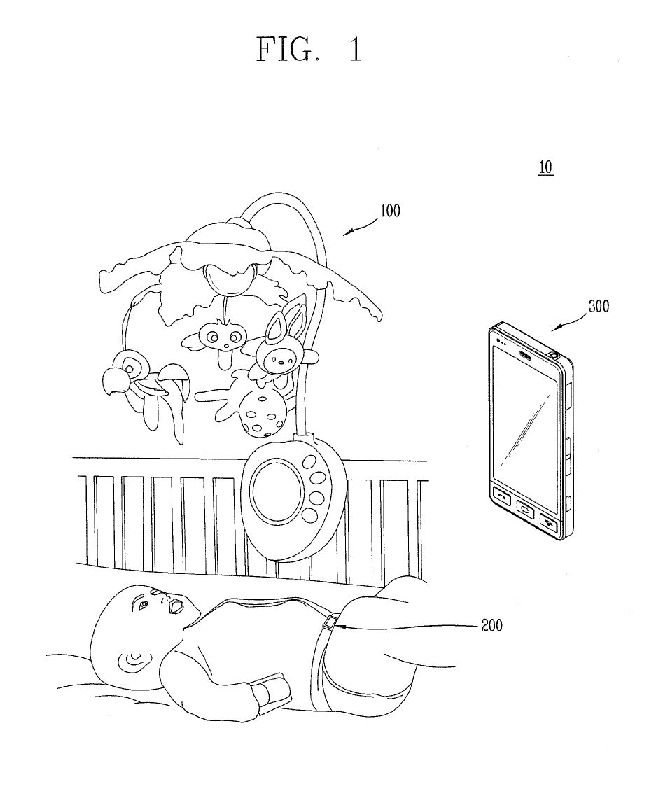

FIG. 1 is a view illustrating the configuration of a health monitoring system according to an exemplary embodiment of the present invention.

The health monitoring system according to an exemplary embodiment of the present invention may include a health monitoring apparatus 100, a sensing device 200, and an external terminal 300.

The health monitoring apparatus 100 may monitor a diaper wearer's physical condition in real time and transmit the monitoring results to the external terminal 300 to inform a guardian (or a caretaker, caregiver, and so on) of the diaper wearer accordingly. To this end, the health monitoring apparatus 100 may analyze an acoustic signal received from the sensing device 200 and determine whether or not any body wastes have been discharged to the diaper based on the analysis results of the acoustic signal.

Also, when it is determined that body wastes have been discharged, the health monitoring apparatus 100 may capture an image of the diaper, analyze the captured image, and transmit information regarding the diaper wearer's physical condition to the external terminal 300.

The sensing device 200 may acquire an acoustic signal generated from the diaper wearer and transmit the acoustic signal acquired for determining whether or not any body wastes have been discharged to the health monitoring apparatus 100. To this end, the sensing device 200 may include a microphone and may have a communication function of transmitting the acoustic signal to the health monitoring apparatus 100.

Meanwhile, the sensing device 200 may have a form which can be detachably attached to a diaper, and may have a sensor to sense whether or not the sensing device 200 is coupled to or separated from the diaper in order to check a point in time at which a diaper is changed. When the sensor senses that the sensing device 200 is separated from the diaper, it may transmit a signal for informing about the separation of the sensing device 200 to the health monitoring apparatus 100. Upon receiving the signal informing about the separation of the sensing device 200 from the diaper from the sensor, the health monitoring apparatus 100 may record a point in time when the signal is received.

The external terminal 300 receives information from the health monitoring apparatus 100, and provides the received information to the guardian of the diaper wearer. To this end, the external terminal 300 may include an interface for providing the received information.

For example, the external terminal 300 may receive a notification message regarding the point in time at which body wastes have been discharged from the health monitoring apparatus 100 and output the received notification message. Also, the external terminal 300 may have an interface for receiving information regarding the diaper wearer's physical condition generated based on characteristic information of an image regarding body wastes from the health monitoring apparatus 100 and providing the received information regarding the diaper wearer's physical condition to the guardian of the diaper wearer.

The health monitoring apparatus 100 and the sensing device 200 may be connected via a short-range communication network so as to transmit and receive signals in real time. Also, the health monitoring apparatus 100 and the external terminal 300 may be connected via a wireless network so as to transmit and receive data in real time or may be connected according to wireless Internet such as Wi-Fi, or the like, or a mobile communication scheme such as GSM, CDMA, or the like.

The sensing device 200 may be a measurement device (or an agent device) in conformity with the communication standards of ISO/IEEE 11073 personal health device (referred to as ‘PHD’, hereinafter) regarding Health Informatics, and the health monitoring apparatus 100 may be a manager device in conformity with communication standards of the PHD.

The sensing device 200 may communicate with the health monitoring apparatus 100 by using a short-range wireless communication scheme. For example, the sensing device 200 may include a communication module in conformity with the Bluetooth standards, and in this case, the sensing device 200 may be implemented to follow Bluetooth health device profile (HDP).

In another example, the sensing device 200 may be implemented to follow a Personal, Home, Hospital Care (PHHC) profile as a communication module in conformity with ZigBee standards, or may include a communication module implemented to follow a Personal Health Device Class (PHDC) as a communication module in conformity with universal serial bus (USB) standards.

Meanwhile, the external terminal 300 may include a mobile phone, a smartphone, a notebook computer, a laptop computer, a digital broadcast terminal, a Personal Digital Assistant (PDA), a Portable Multimedia Player (PMP), a navigation device, and the like. It would be understood by a person in the art that the configuration according to the embodiments of the present disclosure can be also applicable to the fixed types of terminals such as digital TVs, desk top computers, or the like.

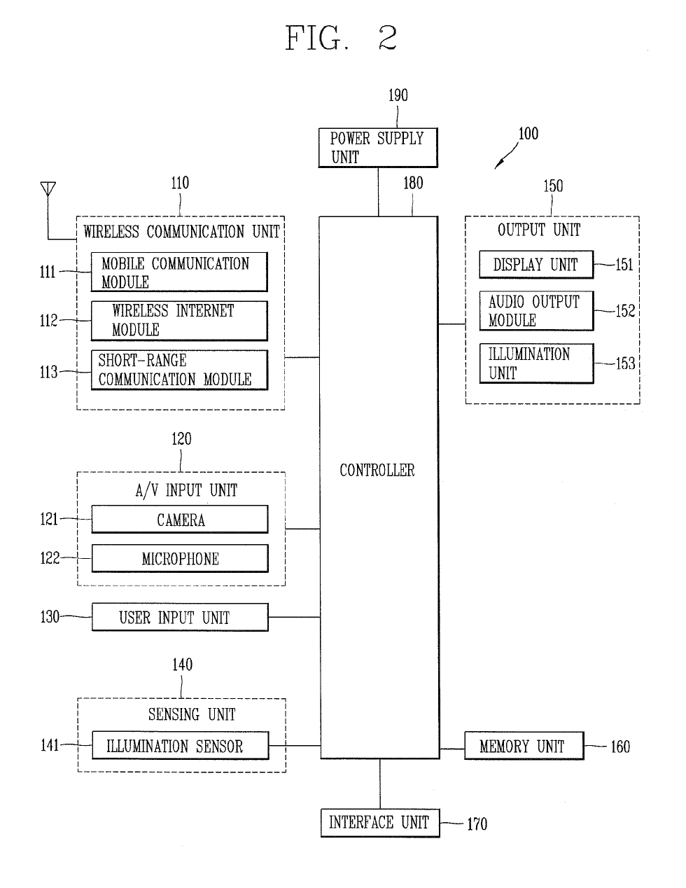

FIG. 2 is a schematic block diagram of a sensing device according to an exemplary embodiment of the present invention.

As shown in FIG. 2, the health monitoring apparatus 100 may include a wireless communication unit 110, an A/V (Audio/Video) input unit 120, a user input unit 130, a sensing unit 140, an output unit 150, a memory 160, an interface unit 170, a controller 180, and a power supply unit 190, and the like. FIG. 2 shows the health monitoring apparatus 100 as having various components, but it should be understood that implementing all of the illustrated components is not a requirement. Greater or fewer components may alternatively be implemented.

The wireless communication unit 110 may include one or more components allowing for radio communication between the health monitoring apparatus 100 and a wireless communication system or between the health monitoring apparatus 100 and a network in which the health monitoring apparatus 100 is located. For example, the wireless communication unit 110 may include a mobile communication module 111, a wireless Internet module 112, a short-range communication module 113, and the like.

The mobile communication module 111 transmits and/or receives radio signals to and/or from at least one of a base station (e.g., access point, Node B, etc.), an external terminal (e.g., other user devices) and a server (or other network entities). Here, the radio signals may include various types of data such as a notification message, characteristic information of an image, a diaper wearer's health information, and the like.

The wireless Internet module 112 refers to a module supporting wireless Internet access for the health monitoring apparatus 100. This module may be internally or externally coupled to the health monitoring apparatus 100. Here, the wireless Internet access technique implemented may include a WLAN (Wireless LAN) (Wi-Fi), Wibro (Wireless broadband), Wimax (World Interoperability for Microwave Access), HSDPA (High Speed Downlink Packet Access), or the like.

The short-range communication module 113 is a module for supporting short range communications. Some examples of short-range communication technology include Bluetooth™, Radio Frequency IDentification (RFID), Infrared Data Association (IrDA), Ultra-WideBand (UWB), ZigBee™, wireless LAN (WLAN), and the like.

The A/V input unit 120 is configured to receive an audio or video signal. The A/V input unit 120 may include a camera 121 (or other image capture device) and a microphone 122 (or other sound pick-up device). The camera 121 processes image data of still pictures or video obtained by an image. The processed image frames may be displayed on a display unit 151 (or other visual output device).

The image frames processed by the camera 121 may be stored in the memory 160 (or other storage medium) or transmitted to the external terminal 300 via the wireless communication unit 110. Two or more cameras 121 may be provided according to the configuration of the health monitoring apparatus 100.

The microphone 122 may receive external sounds (audible data) via a microphone (or the like) and process such sounds into electrical audio data. The processed audio (voice) data may be converted for output into a format transmittable to a mobile communication base station (or other network entity) via the mobile communication module 111. The microphone 122 may implement various types of noise canceling (or suppression) algorithms to cancel (or suppress) noise or interference generated in the course of receiving and transmitting audio signals.

The user input unit 130 (or other user input device) may generate input data for controlling the operation of the health monitoring apparatus 100 by the user. The user input unit 130 may include a keypad, a dome switch, a touch pad (e.g., a touch sensitive member that detects changes in resistance, pressure, capacitance, etc. due to being contacted), a jog wheel, a jog switch, and the like. In particular, the touch pad and the display unit 151 are overlaid in a layered manner to form a touch screen.

The sensing unit 140 detects a current status (or state) of the health monitoring apparatus 100 or a current status of the surroundings of the health monitoring apparatus 100 and generates commands or signals for controlling the operation of the health monitoring apparatus 100. In addition, the sensing unit 140 can detect whether or not the power supply unit 190 supplies power or whether or not the interface unit 170 is coupled with an external device. The sensing unit 140 may include an illumination sensor 141 for sensing brightness of external light.

The output unit 150 is configured to an audio signal or a video signal. The output unit 150 may include the display unit 151, an audio output module 152, an illumination unit 153, and the like.

The display unit 151 may display (output) information processed in the health monitoring apparatus 100. For example, when the health monitoring apparatus 100 is in a setting mode, the display unit 151 may display a User Interface (UI) or a Graphic User Interface (GUI) associated with the setting. When the health monitoring apparatus 100 is in an image capturing mode, the display unit 151 may display a captured image and/or received image, a UI or GUI.

The display unit 151 may include at least one of a Liquid Crystal Display (LCD), a Thin Film Transistor-LCD (TFT-LCD), an Organic Light Emitting Diode (OLED) display, a flexible display, and a three-dimensional (3D) display. Some of them may be configured to be transparent or light-transmissive to allow viewing of the exterior, which may be called transparent displays. A typical transparent display may be, for example, a TOLED (Transparent Organic Light Emitting Diode) display, or the like.

The health monitoring apparatus 100 may include two or more display units (or other display means) according to its particular desired embodiment. For example, a plurality of display units may be separately or integrally disposed on one surface of the health monitoring apparatus 100, or may be separately disposed on mutually different surfaces of the health monitoring apparatus 100.

Meanwhile, when the display unit 151 and a sensor (referred to as a ‘touch sensor’, hereinafter) for detecting a touch operation are overlaid in a layered manner to form a touch screen, the display unit 151 may function as both an input device and an output device. For example, the touch sensor may have a form of a touch film, a touch sheet, a touch pad, and the like.

The touch sensor may be configured to convert pressure applied to a particular portion of the display unit 151 or a change in the capacitance or the like generated at a particular portion of the display unit 151 into an electrical input signal. The touch sensor may be configured to detect the pressure when a touch is applied, as well as the touched position and area. When there is a touch input with respect to the touch sensor, a corresponding signal (signals) is/are transmitted to a touch controller (not shown). The touch controller processes the signals and transmits corresponding data to the controller 180. Accordingly, the controller 180 may recognize which portion of the display unit 151 has been touched.

The audio output module 152 may output audio data received from the wireless communication unit 110 or audio data stored in the memory 160. Also, the audio output module 152 may provide audible outputs related to a particular function performed by the health monitoring apparatus 100. The audio output module 152 may include a speaker, a buzzer, or other sound generating device.

The illumination unit 153 may be configured, for example, as a light emitting diode (LED), and generates light. The illumination unit 153 may be configured to adjust the quantity of light according to an external illuminance (i.e., the quantity of light received by a unit area per unit hour).

The memory 160 may store software programs used for the processing and controlling operations performed by the controller 180, or may temporarily store data (e.g., an image of a diaper, an acoustic signal received from the sensing device 200, or the like) that are inputted or outputted.

The memory 160 may include at least one type of storage medium including a flash memory, a hard disk type, a multimedia card micro type, a card-type memory (e.g., SD or DX memory, etc), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk. Also, the health monitoring apparatus 100 may be operated in relation to a web storage device that performs the storage function of the memory 160 over the Internet.

The interface unit 170 serves as an interface with every external device connected with the health monitoring apparatus 100. For example, the interface unit 170 may include wired or wireless headset ports, external charger ports, wired or wireless data ports, memory card ports, ports for connecting a device having an identification module, audio input/output (I/O) ports, video I/O ports, earphone ports, or the like.

Here, the identification module may be a chip that stores various types of information for authenticating the authority of using the health monitoring apparatus 100 and may include a user identity module (UIM), a subscriber identity module (SIM) a universal subscriber identity module (USIM), and the like.

In addition, the device having the identification module (referred to as ‘identifying device’, hereinafter) may take the form of a smart card. Accordingly, the identifying device may be connected with health monitoring apparatus 100 via a port. The interface may allow for receiving data or power from an external device and delivering the same to each element within the health monitoring apparatus 100 or allow for transmitting data within the health monitoring apparatus 100 to an external device.

When the health monitoring apparatus 100 is connected with an external cradle, the interface unit 170 may serve as a passage to allow power from the cradle to be supplied therethrough to the health monitoring apparatus 100 or may serve as a passage to allow various command signals inputted by the user from the cradle to be transferred to the health monitoring apparatus 100 therethrough. Various command signals or power inputted from the cradle may operate as signals for recognizing that the health monitoring apparatus 100 is properly mounted on the cradle.

The controller 180 typically controls the general operations of the health monitoring apparatus 100. For example, the controller 180 performs controlling and processing to determine whether any body wastes have been discharged or determine health information. The controller 180 may perform a pattern recognition processing to recognize a handwriting input or a picture drawing input performed on the touch screen as characters or images, respectively.

The power supply unit 190 receives external power or internal power and supplies appropriate power required for operating respective elements and components under the control of the controller 180.

The functions of the elements or components applied to the health monitoring apparatus 100 may be implemented within a computer-readable recording medium by using software, hardware, or a combination thereof.

For hardware implementation, the embodiments described herein may be implemented by using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic units designed to perform the functions described herein. In some cases, such embodiments may be implemented by the controller 180 itself. For software implementation, the embodiments such as procedures or functions described herein may be implemented by separate software modules. Each software module may perform one or more functions or operations described herein. Software codes can be implemented by a software application written in any suitable programming language. The software codes may be stored in the memory 160 and executed by the controller 180.

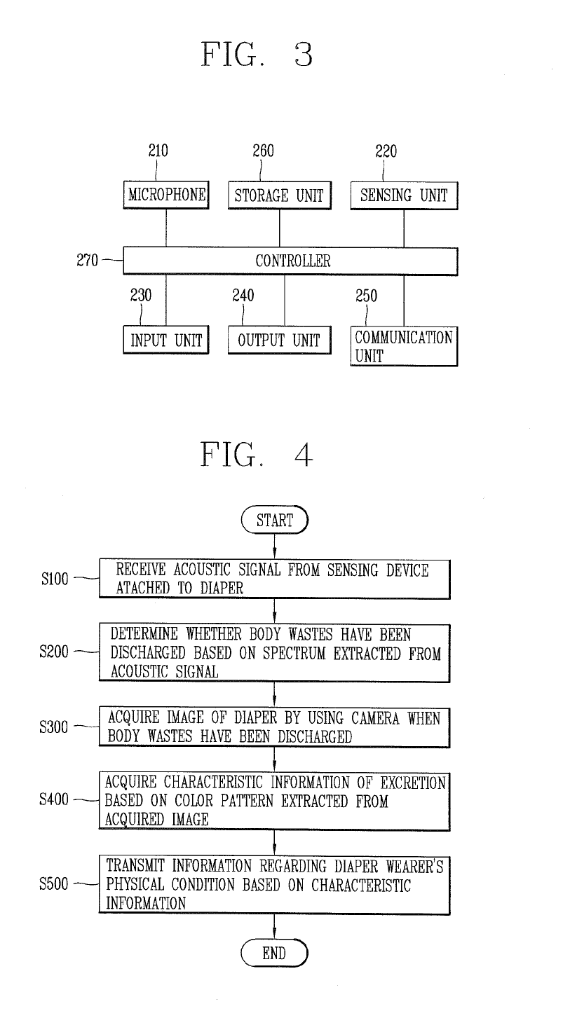

FIG. 3 is a schematic block diagram of a health monitoring apparatus according to an exemplary embodiment of the present invention.

The sensing device 200 may include a microphone 210, a sensing unit 220, an input unit 230, an output unit 240, a communication unit 250, a storage unit 260, and a controller 270.

The microphone 210 receives an external audio signal and processes it into electrical voice data. The processed voice data may be converted into a form transmittable to the health monitoring apparatus 100 via the communication unit 250 and output to the health monitoring apparatus 100.

For example, the audio signal may be expressed in the form in conformity with an ISO/IEEE 11073 Personal Health Device Communication Part 20601: Application Profile—Optimized Exchange Protocol.

The sensing unit 220 may sense (or detect) a current state of the sensing device 200 or a current state in the vicinity of the sensing device 200 and generate a sensing signal for controlling the operation of the sensing device 200.

The sensing unit 220 may include an acceleration sensor (not shown), a gyro sensor (not shown), and a geomagnetic sensor (not shown).

The acceleration sensor is an element for changing a change in acceleration in one direction into an electrical signal. In general, the acceleration sensor may be configured to convert a change in acceleration in three-axis directions regarding a movement of the sensing device 200 into respective electrical signals to measure acceleration of each direction.

The gyro sensor may be a sensor for measuring an angular speed of the sensing device 200 that makes a rotational movement, it can detect a rotated angle with respect to each reference direction. For example, the gyro sensor may detect respective rotated angles, namely, yaw, pitch, and roll, based on the three directional axes.

The geomagnetic sensor may detect azimuth of the sensing device 200 by using magnetic field information formed horizontally on the surface of the ground.

The input unit 230 generates input data for controlling the operation of the sensing device 200 by the user. The user input unit 130 may be configured as a keypad, a dome switch, a touch pad (e.g., a touch sensitive member that detects changes in resistance, pressure, capacitance, etc. due to being contacted), a jog wheel, a jog switch, and the like. In particular, when the touch pad and a display unit (not shown) of the output unit 240 are overlaid in a layered manner, it may be called a touch screen.

The output unit 240 may output an audio signal or a video signal. The output unit 240 may include a display unit, an audio output module (not shown), or the like.

The display unit displays and outputs information processed by the sensing device 200. For example, when the sensing device 200 is a setting mode, the display unit displays a UI (User Interface) or a GUI (Graphical User Interface) related to a setting.

The audio output module outputs audio data stored in the storage unit 260. Also, the audio output module outputs an audio signal related to the function performed by the sensing device 200.

The communication unit 250 may include one or more elements for performing radio communication between the sensing device 200 and a wireless communication system or radio communication between the sensing device 200 and a network in which the sensing device 200 is located.

The controller 260 generally controls a general operation of the sensing device 200. For example, the controller 260 may perform controlling and processing for transmission and reception of an audio signal.

The storage unit 270 may store a program for operating the controller 260 and temporarily store input/output data (e.g., an audio signal, etc.)

FIG. 4 is a flow chart illustrating a health monitoring process of the health monitoring apparatus according to an exemplary embodiment of the present invention.

The wireless communication unit 110 may receive an acoustic signal from the sensing device 200 attached to a diaper (step S100). The wireless communication unit 110 may receive an acoustic signal in real time from the sensing device 200. Also, the controller 180 may store the acoustic signal in the memory 160.

The controller 180 may determine whether or not any body wastes have been discharged based on a spectrum extracted from the acoustic signal received from the sensing device 200 (step S200). The controller 180 detects the frequency of the acoustic signal received in real time from the sensing device 200, and when the detected frequency is higher than a critical frequency, the controller 180 records a time stamp indicating a start point of the excretion (or body wastes). Also, when the detected frequency is continuously lower than the critical frequency during a certain period of time, the controller records a time stamp indicating an end point of the body wastes.

When the time stamp indicating the end point of the body wastes is recorded, the controller 180 extracts a wideband spectrum from the acoustic signal stored in the memory 160 between the start point and end point of the body wastes. Also, the controller 180 determines whether or not body wastes have been discharged based on a distribution range of a basic frequency on the wideband spectrum. For example, when the distribution range of the basic frequency corresponds to a certain range, the controller 180 determines that body wastes have been discharged.

Or, when the time stamp indicating the end point of the body wastes, the controller 180 extracts a narrowband spectrum from the acoustic signal stored in the memory 160 between the start point and the end point of the body wastes. Also, the controller 180 determines whether or not body wastes have been discharged based on a distribution range of a maximum frequency on the narrowband spectrum. For example, when the distribution range of the maximum frequency corresponds to a certain range, the controller 180 determines that body wastes have been discharged.

When it is determined that body wastes have been discharged, the camera 121 may capture an image of a diaper (step S300). For example, the camera 121 may capture an image of a diaper positioned in front of the health monitoring apparatus 100.

In an exemplary embodiment, the wireless communication unit 110 may receive a trigger signal regarding the image capturing from the sensing device 200. In this case, when the trigger signal regarding the image capturing is received from the sensing device 200, the controller 180 may start capturing an image of a diaper positioned in front side of the health monitoring apparatus (100).

Also, the wireless communication unit 110 may transmit information regarding the point in time at which the trigger signal was received to the external terminal 300. The sensing unit 220 of the sensing device 200 may detect whether or not the sensing device 200 is separated from the diaper. Accordingly, when the diaper is changed, the trigger signal is generated by the sensing device 200, and the wireless communication unit 110 may receive the generated trigger signal from the sensing device 200. Accordingly, the diaper change time can be automatically recorded in the external terminal 300.

Or, when the input unit 230 in the sensing device 200 receives an input for applying a trigger signal, for example, when the user manually applies a trigger signal in changing a diaper, the wireless communication unit 110 may receive the trigger signal from the sensing device 200 and transmit information regarding a point in time at which the trigger signal was received to the external terminal 300.

The illumination sensor 141 senses (or detects) ambient illuminance (or intensity of illumination) information, and the controller 180 may control the illumination unit 153 based on the detected illuminance information. The controller 180 may regulate the quantity of light generated from the illumination unit 153 to uniformly maintain the ambient illuminance. Accordingly, the image capture environment with respect to the diaper positioned in front side of the health monitoring apparatus 100 can be uniformly maintained.

When it is determined that body wastes have been discharged, the wireless communication unit 110 may transmit a message informing that body wastes have been discharged to the external device 300. When it is determined that body wastes have been discharged, the controller 180 may generate a message informing that body wastes have been discharged and control the wireless communication unit 110 to transmit the generated message to the external terminal 300.

The controller 180 may acquire characteristic information of the body wastes based on a color pattern extracted from the acquired image (step S400). The controller 180 detects an area of the body wastes through an edge extraction or a pattern extraction from the acquired image. Also, the controller 180 may acquire RGB values by pixel or by pixel area from the detected area of the body wastes and analyze the distribution of the RGB values.

The controller 180 may determine whether or not the body wastes is excrements or urine based on a color contrast or turbidity of the detected area of the body wastes. In general, the diaper may be made of a material that reflects a large quantity of light. Accordingly, when excrements are detected from the diaper, there is a difference in a color contrast between the area of the excrements and an area other than the area of the excrements, and when urine is detected from the diaper, there is a difference in a color contrast between the area of the urine and an area other than the area of the urine, so the controller 180 can determine whether or not the body wastes are excrements or urine based on the difference in a color contrast.

Similarly, when excrements are detected from the diaper, there is a difference in turbidity between the area of the excrements and an area other than the area of the excrements, and when urine is detected from the diaper, there is a difference in turbidity between the area of the urine and an area other than the area of the urine, so the controller 180 can determine whether or not the body wastes are excrements or urine based on the difference in turbidity.

The controller 180 may transmit information regarding the diaper wearer's physical condition to the external terminal based on the characteristic information (step S500). The controller 180 may determine the distribution of the RGB values with respect to the area of the body wastes analyzed in step S400 and patterns of the RGB values consistent within a critical range and read information regarding the diaper wearer's physical condition from the memory 160 according to the determined patterns of the RGB values.

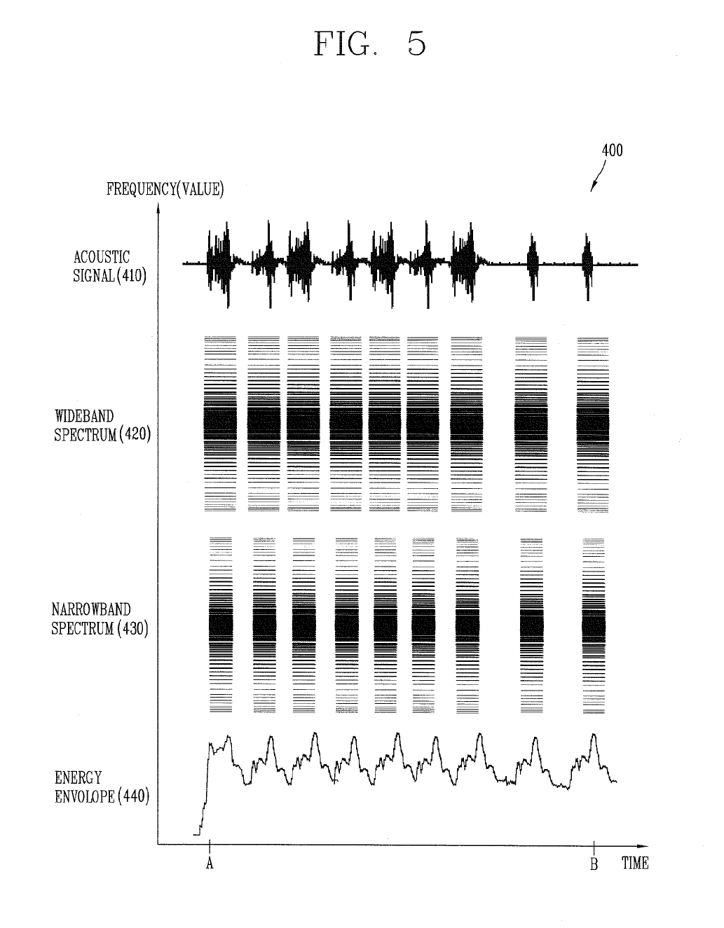

FIG. 5 is a view for explaining spectrum information extracted from an acoustic signal received by the health monitoring apparatus according to an exemplary embodiment of the present invention from the sensing device.

An acoustic signal analysis graph 400 represents analysis results of the acoustic signal received by the health monitoring apparatus (100) in real time from the sensing device 200. In the acoustic signal analysis graph 400, time by ms corresponds to the X axis and values of frequency 410, a wideband spectrum 420, a narrowband spectrum 430 (Hz unit), and energy envelope 440 of an acoustic signal correspond to the Y axis.

The controller 180 may sample the acoustic signal received in real time from the sensing device 200 by using, for example, 24 KHz of 16 bits and store the same in the memory 160. The controller 180 detects the frequency from the acoustic signal in real time, and when the detected frequency is higher than a critical frequency (which is determined by experiments to be the lowest frequency of the acoustic signal detected in the occurrence of body wastes), the controller 180 records a time stamp (A).

Also, the controller 180, while storing the acoustic signal in the memory 160, detects the frequency from the acoustic signal in real time, and when the detected frequency is continuously lower than the critical frequency during a certain period of time (which is determined by experiments to be a maximum period of time during which the frequency lower than the critical frequency is continuously detected in the occurrence of body wastes), the controller 180 records a time stamp (B).

When the time stamp (B) is recorded, the controller 180 analyzes an acoustic signal between the time stamp (A) and the time stamp (B). For example, the controller 180 extracts a wideband spectrum from the acoustic signal between the time stamp (A) and the time stamp (B) and analyzes the extracted wideband spectrum. The controller 180 analyzes a distribution range of a basic frequency of the extracted wideband spectrum, and when the distribution range corresponds to a certain range, the controller 180 may determine that body wastes have been discharged. For example, the certain range may be 67 to 125 Hz. Also, in this case, the controller 180 may analyze the wideband spectrum in a portion (e.g., an initial section corresponding to one-third of the entire section) of the section between the time stamp (A) and the time stamp (B).

Or, the controller 180 extracts the narrowband spectrum from the acoustic signal between the time stamp (A) and the time stamp (B) and analyzes the extracted wideband spectrum. The controller 180 may analyze a distribution range of a maximum frequency of the extracted wideband spectrum, and when the distribution range corresponds to a certain range, the controller 180 may determine that body wastes have been discharged. For example, the certain range may be 4 to 12 KHz.

In a different exemplary embodiment, the controller 180 may perform an envelop checking on an acoustic signal. For example, the controller 180 may determine whether or not body wastes have been discharged by analyzing morphologic factors of a hamming window in the unit of 1 ms formed from the acoustic signal.

Meanwhile, with respect to the acoustic signal received in real time from the sensing device 200, the controller 180 may perform sampling thereon by using, for example, 8 KHz of 16 bits, and store the same in the memory 160. The controller 180 detects the frequency from the acoustic signal in real time, and when the detected frequency is higher than a critical frequency (which is determined by experiments to be the lowest frequency of the acoustic signal detected when baby formula or mother's milk is swallowed by the baby), the controller 180 records a time stamp (A).

Also, the controller 180, while storing the acoustic signal in the memory 160, detects the frequency from the acoustic signal in real time, and when the detected frequency is continuously lower than the critical frequency during a certain period of time (which is determined by experiments to be a maximum period of time during which the frequency lower than the critical frequency is continuously detected when baby formula or mother's milk are swallowed by the baby), the controller 180 records a time stamp (B).

When the time stamp (B) is recorded, the controller 180 analyzes an acoustic signal between the time stamp (A) and the time stamp (B). For example, the controller 180 extracts a narrowband spectrum from the acoustic signal between the time stamp (A) and the time stamp (B) and analyzes the extracted narrowband spectrum. The controller 180 analyzes a distribution range of a maximum frequency of the extracted narrowband spectrum, and when the distribution range corresponds to a certain range, the controller 180 may determine that baby formula or mother's milk are swallowed by the baby. For example, the certain range may be 1 KHz or lower.

The controller 180 may count the number of times that the baby formula or mother's milk have been swallowed by the baby between the time stamp (A) and the time stamp (B), and store a resultant value obtained by multiplying the quantity of baby formula or mother's milk that can be taken when the baby formula or mother's milk are swallowed by the baby one time by gender/age of the feeder of the baby formula or the mother's milk to the number of times that baby formula or mother's milk were swallowed by the baby, in the memory 160. Also, the controller 180 may transmit the result value stored in the memory 160 to the external terminal 300.

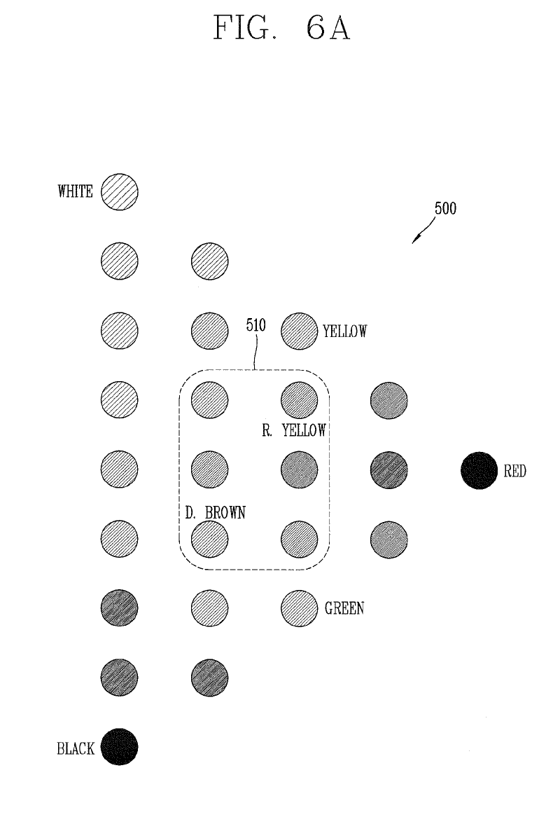

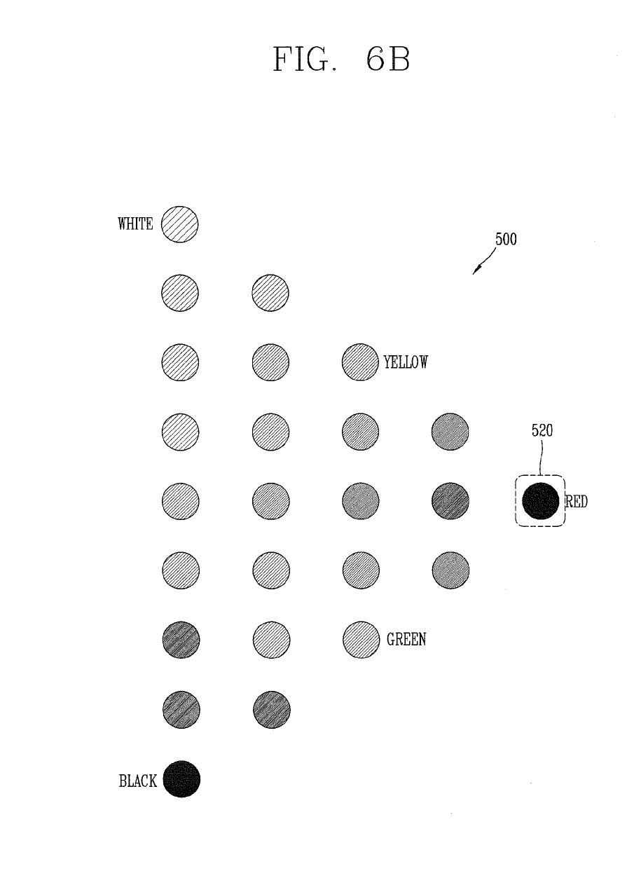

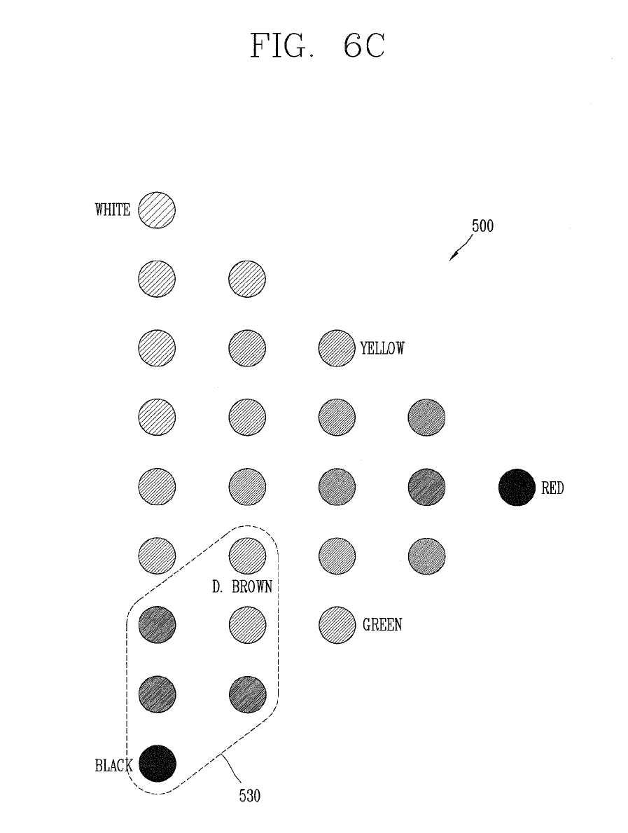

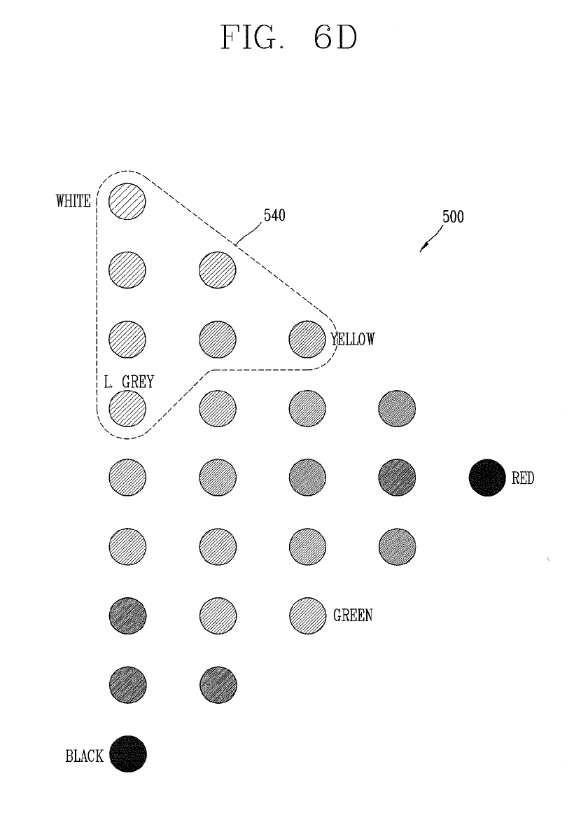

FIGS. 6A to 6D are views for explaining color patterns extracted from images obtained by the health monitoring apparatus according to an exemplary embodiment of the present invention.

As illustrated, a color distribution 500 may have an equilateral triangle with white, black, and red elements as vertexes. A yellow element may be positioned in the middle between the white and red elements, a green element may be positioned in the middle between red and black elements. Other elements may have a color in a mixture ratio of the five elements according to their positions on the color distribution 500.

FIG. 6A illustrates a distribution of the colors of excrement of a healthy person according to an exemplary embodiment of the present invention.

When a color pattern of an area of body wastes detected from an image obtained with respect to a diaper corresponds to a range 510 from reddish yellow to dark brown, it may be determined that a diaper wearer's physical conditions is good.

The controller 180 may determine the intake of nutritive elements of the diaper wearer based on the density of the color pattern of the area of the body wastes (e.g., it is determined that as the density of the color pattern of the area of the body wastes is dark, the intake of nutritive elements is large, and as the density of the color pattern of the area of the body wastes is light, the intake of nutritive elements is small). Also, the controller 180 may determine foods ingested by the diaper wearer based on the color pattern of the area of the detected body wastes.

FIG. 6B illustrates a distribution of the colors of bloody excrements according to an exemplary embodiment of the present invention.

When a color pattern of an area of body wastes detected from an image obtained with respect to the diaper positioned in front side of the health monitoring apparatus 100 corresponds to red color 520, it may be determined that a diaper wearer's physical conditions is not good. In this case, the health monitoring apparatus 100 may transmit information regarding a suspected disease (cholera, food poisoning, etc.) or an ailment (colon cancer, cancer of the rectum, etc.) of the diaper wearer to the external terminal 300 along with a warning message indicating that the diaper wearer must be carefully watched and must immediately see the doctor.

FIG. 6C illustrates a distribution of constipation colors according to an exemplary embodiment of the present invention.

When a color pattern of an area of body wastes detected from an image obtained with respect to the diaper corresponds to a range 530 from dark brown to black, it may be determined that a diaper wearer is constipated. The controller 180 may determine a period of time during which the diaper wearer's excrement has been stayed in his intestine based on the density of the color pattern of the area of the excrement. Also, the controller 180 may store information indicating that the diaper's wearer is constipated in the memory 160 and calculate the period of time in which the diaper's wearer is in the state of being constipated. When it is determined that the diaper wearer is constipated for a long time, the controller 180 may transmit information indicating that the diaper wearer is constipated along with a warning message indicating that the diaper wearer must be carefully watched and must immediately see the doctor.

FIG. 6D illustrates a distribution of colors of excrement of under nourishment according to an exemplary embodiment of the present invention.

When a color pattern of an area of body wastes detected from an image obtained with respect to the diaper corresponds to a range 540 from pale gray to pale greenish yellow, it may be determined that a diaper wearer is malnourished.

The controller 180 may determine the intake of nutritive elements of the diaper wearer based on the density of the color pattern of the area of the body wastes (e.g., it is determined that as the density of the color pattern of the area of the body wastes is dark, the intake of nutritive elements is large, and as the density of the color pattern of the area of the body wastes is light, the intake of nutritive elements is small).

In this case, the health monitoring apparatus 100 may transmit information regarding a suspected disease (acute hepatitis, when there is no problem after valium checkup) or an ailment (cancer of the pancreas, intestinal tuberculosis, etc.) of the diaper wearer to the external terminal 300 along with a warning message indicating that the diaper wearer must be carefully watched and must immediately see the doctor.

The controller 180 may control the communication unit 110 to transmit information regarding the determined physical condition (namely, disease or ailment information, information regarding a required measure to be taken, or the like) to the external terminal 300.

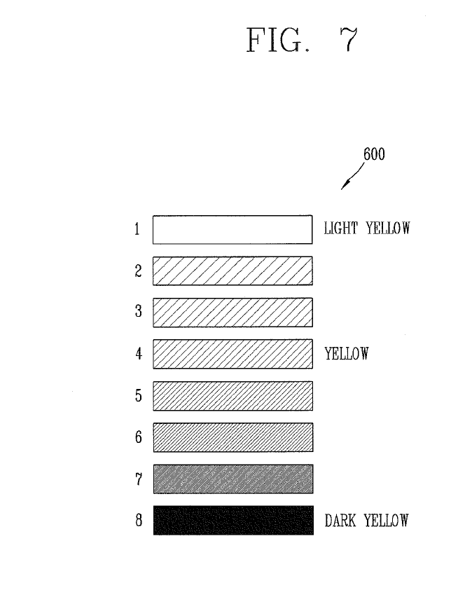

FIG. 7 is a view for explaining a color pattern of urine extracted from an image obtained by the health monitoring apparatus according to an exemplary embodiment of the present invention.

In a color distribution 600, a light yellow element is positioned atop, a dark yellow element is positioned at the bottom, and a yellow element is positioned in the middle. The other elements denote mixture colors of the three elements according to their positions. The elements are discriminated by eight steps, and level 1 is given to the light yellow element positioned atop and level 8 is given to the dark yellow element positioned at the bottom.

When a color pattern of an area of body wastes detected from an image obtained with respect to the diaper corresponds to the level 1 or level 2, the controller 180 determines that body moisture content is sufficient and there is no change in weight. When a color pattern of an area of body wastes detected from an image obtained with respect to the diaper corresponds to the level 3 or level 4, the controller 180 may determine that there is a little dehydration and there has been a weight loss of 1 to 3%.

Also, when a color pattern of an area of body wastes detected from an image obtained with respect to the diaper corresponds to the level 5 or level 6, the controller 180 may determine that there is severe dehydration and there has been a weight loss of 5% or greater.

The controller 180 may control the communication unit 110 to transmit information regarding the determined physical condition (namely, disease or ailment information, information regarding a required measure to be taken, or the like) to the external terminal 300.

Also, when a color pattern of an area of body wastes detected from an image obtained with respect to the diaper is slightly yellow urine, the controller 180 may determine that the urine is healthy urine and also determine a water intake according to the concentration of the color pattern.

Also, the controller may determine water intake based on the concentration of a color pattern of an area of body wastes detected from an image obtained with respect to the diaper, and may determine a numerical value of uric acid, whether there is a kidney stone, whether there is a bacterial infection of a urological system, or the like.

Also, the controller 180 may determine a change in weather or the type and amount of ingested food, and determine a cause of a change in color, according to a color pattern of an area of body wastes detected from an image obtained with respect to the diaper.

Some examples of the causes of the change in color may include: consumption of red turnips, blackberries, and foods containing food coloring(s); when medicine such as phenytoin, or the like, is taken; when there is a disease such as porphyrinemia; when there is phenolphthalein poisoning; and when urate crystals, red diaper syndrome, existence of blood, hemoglobin, myoglobulin in urine, or the like, is detected. When there is porphyrinemia, urine having red wine color is detected. When riboflavin is taken, urine having yellow color is detected. When urine contains urate crystals or when medicine such as Rifapin, or the like, is taken, an orange color is detected.

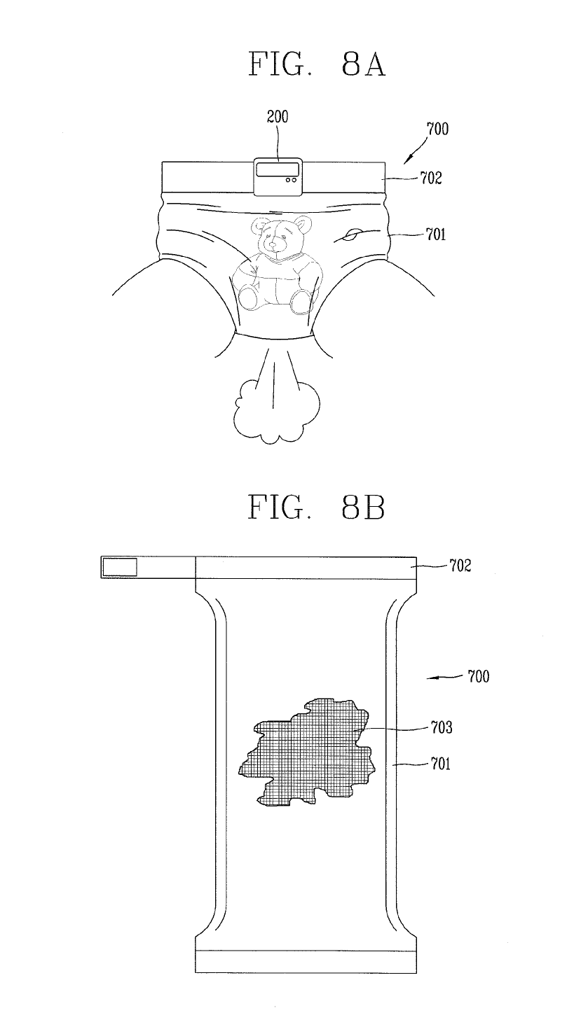

FIG. 8A is a view for explaining a process of receiving an acoustic signal or a trigger signal from the sensing device by the health monitoring apparatus according to an exemplary embodiment of the present invention.

A diaper 700 may include a body part 701 and a band part 702. The sensing device 200 may be attached to the body part 702 or the band part 702 of the diaper 700. The sensing device 200 may acquire an acoustic signal generated in the vicinity of the sensing device 200 in real time through a microphone 210. Also, the sensing device 200 may transmit the acquired acoustic signal to the health monitoring apparatus 100 through the communication unit 250.

The sensing device 200 may receive a trigger signal indicating a change of diaper through the input unit 230. For example, when the diaper is to be changed, the user may apply a trigger signal indicating a change of a diaper to the sensing device 200 through a button provided on the input unit 230. Accordingly, the sensing device 200 may transmit the received trigger signal to the health monitoring apparatus 100 through the communication unit 250.

Or, the sensing device 200 may detect the trigger signal indicating a change of a diaper through the sensing unit 220. For example, the sensing device 200 may be configured to have a clip type and may be attached such that two opposing faces of the clip are brought into contact with outer and inner faces of the band 702 of the diaper 700. In the state in which the sensing device 200 is attached to the diaper 700, when the sensing device 200 is separated from the diaper 700 and detects that two opposing faces of the clip come into contact, the sensing unit 220 may generate a trigger signal. Accordingly, the sensing device 200 may transmit the generated trigger signal to the health monitoring apparatus 100 through the communication unit 250.

FIG. 8B is a view for explaining a process of analyzing a color pattern from an image of a diaper according to an exemplary embodiment of the present invention.

When the health monitoring apparatus 100 determines that body wastes have been discharged, or when the health monitoring apparatus 100 receives a trigger signal from the sensing device 200, the health monitoring apparatus 100 acquires an image of the diaper through the camera 121. Also, the controller 180 may detect an area 703 of body wastes of the diaper 700 from the acquired image through an edge extraction or a pattern extraction.

Also, the controller 180 may acquire RGB values in the detected area 703 of body wastes by pixel or by pixel area, and analyze the distribution of the RGB values. Also, the controller 180 determines the pattern of the RGB values consistent in a critical range, and reads the user's health information from the memory 160 according to the determined pattern of the RGB values.

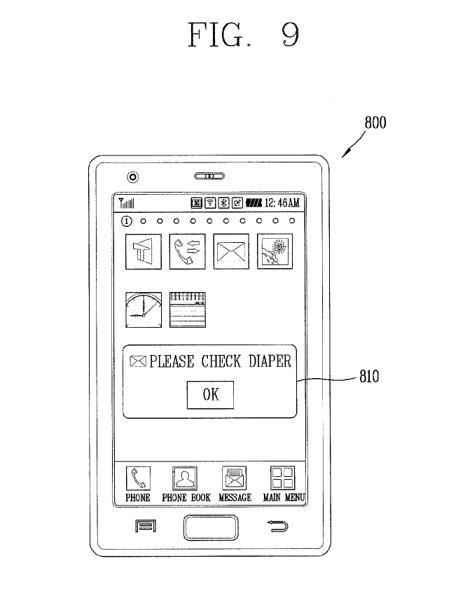

FIG. 9 is an overview of a display screen illustrating a transmission of a message indicating that body wastes have been discharged to an external terminal by the health monitoring apparatus according to an exemplary embodiment of the present invention.

The health monitoring apparatus 100 determines whether or not body wastes have been discharged. When the health monitoring apparatus 100 determines that body wastes have been discharged, it may generate a message indicating that body wastes have been discharged, and transmit the generated message to the external terminal 300. Accordingly, the external terminal 300 may output the message received from the health monitoring apparatus 100.

As shown in FIG. 9, when the external terminal 300 receives a message from the health monitoring apparatus 100, it may display a notification window 810 including the received message on a screen 800. Or, when the external terminal 300 receives a message from the health monitoring apparatus 100, it can output the received message in an audible manner or in a tactile manner. For example, the external terminal 300 may output a message ‘Please check diaper’ by voice, or may generate vibration reflecting content of the received message.

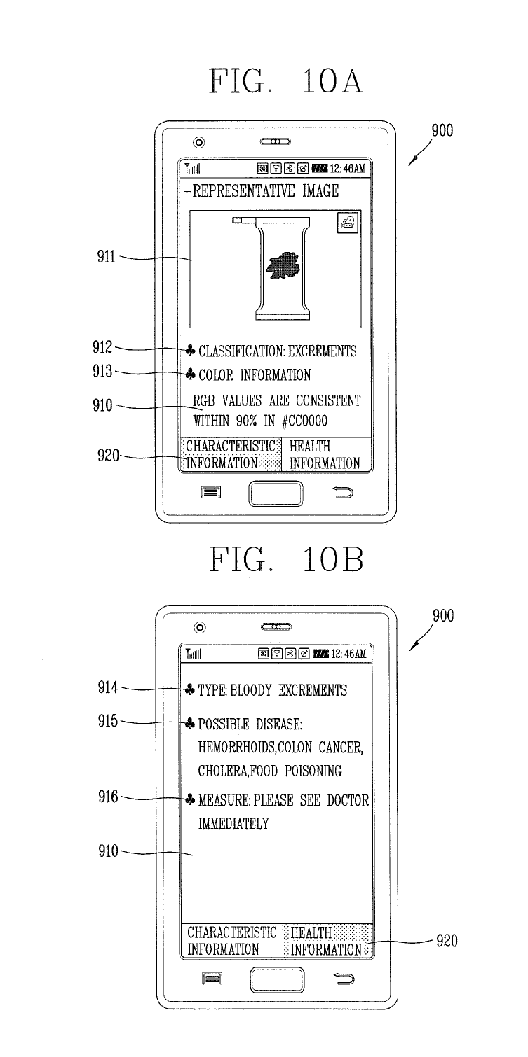

FIGS. 10A and 10B are overviews of display screens illustrating the process of transmitting health information to the external terminal by the health monitoring apparatus according to an exemplary embodiment of the present invention.

The health monitoring apparatus 100 may transmit information regarding the diaper wearer's physical condition to the external terminal 300 based on characteristic information extracted from the image of the diaper. Also, the health monitoring apparatus 100 may transmit the characteristic information to the external terminal 300.

With reference to FIG. 10A, the external terminal 300 provides an interface 900 indicating the characteristic information extracted from the image of the diaper received from the health monitoring apparatus 100. The interface 900 displaying the characteristic information includes an information display area 910 and a menu area 920. When a characteristic information tap is selected from the menu area 920, the characteristic information received from the health monitoring apparatus 100 is displayed on the information display area 910.

The information display area 910 may display at least one image, e.g., a representative image 911 having the highest definition of the area of the body wastes, captured after the excretion, a type of the body wastes (excrements or urine) 910, and color information 913 extracted from the image.

With reference to FIG. 10b, the external terminal 300 provides the interface 900 displaying information regarding the diaper wearer's physical condition extracted from the image of the diaper received from the health monitoring apparatus 100. The interface 900 displaying characteristic information includes an information display area 910 and a menu area 920. When a characteristic information tap is selected from the menu area 920, the information regarding the diaper wearer's physical condition received from the health monitoring apparatus 100 is displayed on the information display area 910.

The information display area 910 may display a type of excrements or urine (e.g., the type of excrements or urine described above with reference to FIGS. 6A to 6D or 7) 914, disease information 915, and measures to be taken 916 regarding the health information. Besides, the information display area 910 may further display information regarding a cause of a disease or an ailment, or information regarding the diaper wearer's physical condition determined based on the color of the excrements or urine, such as food ingestion information, or the like.

According to an exemplary embodiment of the present invention, the health monitoring apparatus automatically detects whether or not diaper wearer, who is not able to move freely, have discharged body wastes based on spectrum information extracted from an acoustic signal received from the sensing device attached to the diaper, and provides corresponding information to the external device, to thus allow the guardian of the diaper wearer to conveniently monitor the diaper wearer's physical condition.

Also, the health monitoring apparatus captures an area of body wastes, and conveniently monitors the diaper wearer's physical condition based on a color pattern through image processing.

In the embodiments of the present invention, the above-described method can be implemented as codes that can be read by a computer in a program-recorded medium. The computer-readable medium includes various types of recording devices in which data read by a computer system is stored. The computer-readable medium may include a ROM, a RAM, a CD-ROM, a magnetic tape, a floppy disk, an optical data storage device, and the like. The computer-readable medium also includes implementations in the form of carrier waves or signals (e.g., transmission via the Internet). The health monitoring apparatus and the health monitoring method according to the embodiments of the present disclosure is not limited in its application of the configurations and methods, but the entirety or a portion of the embodiments can be selectively combined to be configured into various modifications.

First, terms or words used in the present specification and the claims should not be construed as being limited to general or literal meaning, but should be construed in meaning and concept that coincide with the technical spirit of the invention. Thus, configurations shown in embodiments and the drawings of the present invention rather is an example of the most exemplary embodiment and does not represent all of the technical spirit of the invention. Thus, it will be understood that various equivalents and modifications that replace the configurations are possible when filing the present application.

What is claimed is:

1. A health monitoring method of a health monitoring apparatus, the method comprising:
receiving an acoustic signal from a sensing device attached to a diaper;
determining whether any body wastes have been discharged based on a spectrum extracted from the acoustic signal;
when it is determined that body wastes have been discharged, obtaining an image of the diaper by using a camera;
obtaining characteristic information of the body wastes based on a color pattern extracted from the obtained image; and
transmitting information regarding the diaper wearer's physical condition to an external terminal based on the characteristic information.

2. The method of claim 1, wherein the spectrum comprises a wideband spectrum and/or narrowband spectrum extracted from the acoustic signal.

3. The method of claim 1, wherein, in determining whether any body wastes have been discharged, whether any body wastes have been discharged is determined based on a distribution range of a basic frequency on the wideband spectrum.

4. The method of claim 2, wherein, in determining whether any body wastes have been discharged, whether any body wastes have been discharged is determined based on a distribution range of a maximum frequency of the narrowband spectrum.

5. The method of claim 1, further comprising:
receiving a trigger signal from the sensing device,
wherein, in obtaining the image of the diaper, the image of the diaper is obtained based on the trigger signal.

6. The method of claim 5, further comprising:
transmitting information regarding a point in time at which the trigger signal was received to the external terminal.

7. The method of claim 5, wherein the trigger signal is generated when the sensing device is separated from the diaper.

8. The method of claim 1, wherein the obtaining of the characteristic information of body wastes comprises:
detecting an area corresponding to the diaper from the obtained image; and
determining whether or not the body wastes are excrements or urine based on a color contrast or turbidity of the detected area.

9. The method of claim 1, wherein the obtaining of the image comprises:
detecting illuminance information of the surroundings; and
controlling illumination based on the detected illuminance information.

10. The method of claim 1, wherein the obtaining of the image comprises:
when it is determined that body wastes have been discharged, transmitting a message informing that body wastes have been discharged to the external terminal.

11. A health monitoring apparatus comprising:
a communication unit configured to receive an acoustic signal from a sensing device attached to a diaper;
a camera configured to acquire an image of the diaper; and
a controller configured to determine whether or not any body wastes have been discharged based on a spectrum extracted from the acoustic signal, control the camera to acquire the image when it is determined that body wastes have been discharged, obtain characteristic information of the body wastes based on a color pattern extracted from the obtained image, and control the communication unit to transmit obtained information regarding the diaper wearer's physical condition to an external terminal based on the characteristic information.

12. The apparatus of claim 11, wherein the spectrum may include a wideband spectrum and/or narrowband spectrum extracted from the acoustic signal.

13. The apparatus of claim 12, wherein the controller determines whether any body wastes have been discharged, based on a distribution range of a basic frequency on the wideband spectrum.

14. The apparatus of claim 12, wherein the controller determines whether any body wastes have been discharged is determined, based on a distribution range of a maximum frequency of the narrowband spectrum.

15. The apparatus of claim 11, wherein the communication receives a trigger signal from the sensing device, and the controller controls the camera to obtain the image based on the trigger signal.

16. The apparatus of claim 15, wherein the communication unit transmits information regarding a point in time at which the trigger signal was received to the external terminal.

17. The apparatus of claim 15, wherein the trigger signal is generated when the sensing device is separated from the diaper.

18. The apparatus of claim 11, wherein the controller detects an area corresponding to the diaper from the obtained image, and determines whether or not the body wastes are excrements or urine based on a color contrast or turbidity of the detected area.

19. The apparatus of claim 11, further comprising:
an illumination unit providing illumination by using an illumination source; and
an illuminance sensing device configured to detect illuminance information of surroundings,
wherein the controller controls the illumination provided by the illumination unit based on the detected illuminance information.

20. The apparatus of claim 11, wherein when it is determined that body wastes have been discharged, the controller controls the communication unit to transmit a message informing that body wastes have been discharged, to the external terminal.
